(19) United States
(12) Patent Application Publication
(10) Pub. No.: US 2022/0096850 A1
(43) Pub. Date: Mar. 31, 2022

(54) DEPLOYABLE STRUCTURES TO ANCHOR IMPLANTED DEVICES

(71) Applicant: MEDTRONIC, INC., Minneapolis, MN (US)

(72) Inventors: Michael E. Winek, Minneapolis, MN (US); Lauren E. Barth, Minneapolis, MN (US); Lucine Bell N. Solefack, Inver Grove Heights, MN (US)

(21) Appl. No.: 17/039,732

(22) Filed: Sep. 30, 2020

(51) Int. Cl. A61N 1/375

(52) U.S. Cl. CPC A61N 1/37518; A61N 1/0534; A61N 1/3754

(57) ABSTRACT

Implantable medical systems include an infusion or stimulation medical device that may be implanted and anchored within a subcutaneous or subfascial pocket by deployable anchors. The deployable anchors may be retractable. The deployable anchors may be deployed from an integral anchor system of the implanted device or from a separate chassis coupled to the implanted device. Actuators that may be controlled by a clinician may be used to deploy the anchors from a retracted state to a deployed state.

### TECHNICAL FIELD

Deployable structures provide anchoring of implanted devices such as stimulation or infusion devices within a body of a patient.

### BACKGROUND

Implantable medical systems provide medical therapy to patients typically by delivering therapy stimulus to a target area within the body of the patient or delivery of a drug to the target area within the body of the patient. The implantable medical system includes a device that is implanted subcutaneously or subfascially. This provides the convenience of no potentially cumbersome external device while also eliminating the need to create a delivery path that transfers from outside of the body to within the body.

In many cases, the target area for the therapy is not suitable for implantation of the device. The device is implanted at a location of convenience and an implantable medical lead, lead and extension, or catheter, are positioned to extend between the target area and the location where the device is implanted to carry the therapy from the device to the target area.

At the location of convenience, the device is typically implanted within a tissue pocket, formed in the subcutaneous fat layer that lies adjacent to a fascial tissue layer or in other locations such as subfascial positions. To avoid various problems, it is often desirable to fix the position of the device within the tissue pocket by the clinician surgically suturing the device in place by suturing to the fascial tissue. However, there are drawbacks to manually suturing the device within the pocket. For instance, this manual process requires additional manual effort and introduces the potential for human error by the surgeon. Furthermore, this manual process requires the additional time to complete the suturing which lengthens the experience for the patient. Additionally, it relies on the robustness of the suture to secure the device.

### SUMMARY

Embodiments address issues such as these and others by providing structures that may be deployed to anchor the implanted device within the tissue pocket, either to supplement or replace the manual suturing process. Embodiments provide for the deployable structures to be included with the implanted device in various ways. Embodiments provide for many variations in the deployable structures including how the structures are deployed, the shapes of the structures, retractability, and the like as disclosed in more detail herein.

Embodiments provide an anchoring system for an implanted device of an implantable medical system. The anchoring system includes an anchor configured to become affixed to fascial tissue part of or adjacent to a tissue pocket. The anchoring system further includes an actuator coupled to the anchor, the actuator being responsive to a triggering event to move the anchor between a retracted position and a deployed position where the anchor becomes affixed to the fascial tissue once in the deployed position.

Embodiments provide an anchoring system for an implantable medical system that includes an implanted device. The anchoring system includes an anchor configured to become affixed to tissue. The anchoring system also includes an actuator coupled to the anchor. The actuator is responsive to a first triggering event to move the anchor from a retracted position to a deployed position where the anchor becomes affixed to the tissue once in the deployed position. The actuator is responsive to a second triggering event that is subsequent to the first triggering event to move the anchor from the deployed position to the retracted position. The actuator is coupled in a fixed position relative to the implanted device.

Embodiments provide an anchoring system for an implantable medical system. The anchoring system includes an anchor configured to become affixed to fascial tissue adjacent a tissue pocket. The anchoring system includes an actuator coupled to the anchor. The actuator is responsive to a first triggering event to move the anchor from a retracted position to a deployed position where the anchor becomes affixed to the fascial tissue once in the deployed position. The actuator is responsive to a second triggering event that is subsequent to the first triggering event to move the anchor from the deployed position to the retracted position.

### DESCRIPTION OF THE DRAWINGS

FIG. 1 shows an example of an environment for embodiments of anchoring systems utilizing deployable structures.

FIG. 2 shows an example of a tissue pocket within the environment of FIG. 1 where deployable structures of an anchoring system are present.

FIG. 3A shows a front view of a generalized anchoring system integral to an implanted device.

FIG. 3B shows a top view of the generalized anchoring system integral to an implanted device.

FIG. 4A shows a front view of a generalized anchoring system including a chassis mounted to an implanted device.

FIG. 4B shows a top view of the generalized anchoring system including a chassis mounted to an implanted device.

FIG. 5A shows a front view of a generalized anchoring system integral to an implanted device with multiple anchor points provided within the peripheral of the implanted device.

FIG. 5B shows a top view of the generalized anchoring system integral to an implanted device with multiple anchor points provided within the peripheral of the implanted device.

FIG. 6A shows a front view of a generalized anchoring system integral to an implanted device with a centrally located anchor point.

FIG. 6B shows a top view of the generalized anchoring system integral to an implanted device with a centrally located anchor point.

FIG. 7 shows an embodiment of an anchoring system with a chassis attached to an implanted device with retention lips and ratchets.

FIG. 8 shows another embodiment of an anchoring system with a chassis attached to an implanted device with retention lips and ratchets.

FIG. 9 shows an embodiment of an anchoring system that utilizes chambers with wipers for deployable structures.

FIG. 10 shows an embodiment of an anchoring system with deployable spines distributed about the implanted device or separate chassis.

FIG. 11 shows an embodiment of an anchoring system with deployable spines in a central location of the implanted device or separate chassis.

FIG. 12 shows an embodiment of an anchoring system where the spine has an alternative spiral shape.

FIG. 13 shows an embodiment of an anchoring system with a hook-shaped deployable structure that includes a thumb button and associated notches for controlling deployment.

FIG. 14 shows a more detailed view of the thumb button and associated notches of the embodiment of FIG. 13.

FIG. 15 shows an embodiment of an anchoring system that includes a hook-shaped barbless deployable structure.

FIG. 16 shows an embodiment of an anchoring system that includes a hook-shaped barbed deployable structure.

FIG. 17A shows an embodiment of a generalized anchoring system that utilizes barbs that are present on peripheral anchor points and are shown in a retracted state.

FIG. 17B shows the embodiment of FIG. 17A with the barbs in the deployed state.

FIG. 18A shows an embodiment of a generalized anchoring system that utilizes barbs that are present in a central anchor point and are shown in a retracted state.

FIG. 18B shows the embodiment of FIG. 18A where the barbs are a straight shape and are in the deployed state.

FIG. 18C shows the embodiment of FIG. 18A where the barbs are a curved shape and are in the deployed state.

FIG. 19A shows another embodiment of a generalized anchoring system that utilizes barbs that are present in a central anchor point and are shown in a retracted state.

FIG. 19B shows the embodiment of FIG. 19A where the barbs are in the deployed state.

FIG. 20 shows an embodiment of an anchoring system that utilizes a plunger actuator to deploy and/or retract the anchor.

FIG. 21 shows an embodiment of an anchoring system that utilizes a spring actuator to deploy and/or retract the anchor.

FIG. 22 shows an embodiment of an anchoring system that utilizes a magnetic field actuator to deploy and/or retract the anchor.

FIG. 23 shows an embodiment of an anchoring system that utilizes a tool interface actuator to deploy and/or retract the anchor.

FIG. 24 shows an embodiment of an anchoring system that utilizes a rack and worm screw actuator to deploy and/or retract the anchor.

FIG. 25 shows an embodiment of an anchoring system that utilizes a motorized actuator to deploy and/or retract the anchor.

FIG. 26 shows an example of circuitry used to control the motorized actuator of the embodiment of FIG. 25 with an external control signal.

FIG. 27 shows an example of a saber shaped barb that may be used as the deployable anchor in the embodiments of FIGS. 20-25.

FIG. 28 shows an embodiment of a guide provided by a separate structure surrounding the implanted device to keep the lead or catheter outside of the anchor space.

FIG. 29 shows an embodiment of a guide provided as an integral component of the implanted device or separate anchor system chassis to keep the lead or catheter outside of the anchor space.

FIG. 30 shows an embodiment of a guide enclosure provided as an attached or integral component of the implanted device or anchoring system chassis to keep the lead or catheter outside of the anchor space.

FIG. 31 shows a first embodiment of the operations performed when implanting an implanted device and using deployable structures of an anchoring system to anchor the implanted device.

FIG. 32 shows a second embodiment of the operations performed when implanting an implanted device and using deployable structures of an anchoring system to anchor the implanted device.

FIG. 33 shows a third embodiment of the operations performed when implanting an implanted device and using deployable structures of an anchoring system to anchor the implanted device.

### DETAILED DESCRIPTION

Embodiments provide an anchoring system that includes deployable structures that anchor an implanted device within a subcutaneously or subfascially formed pocket within the body of a patient. The deployable structures may pierce into the fascial tissue adjacent to the subcutaneous or subfascial location to fix the location and orientation of the implanted device within the subcutaneous or subfascial pocket.

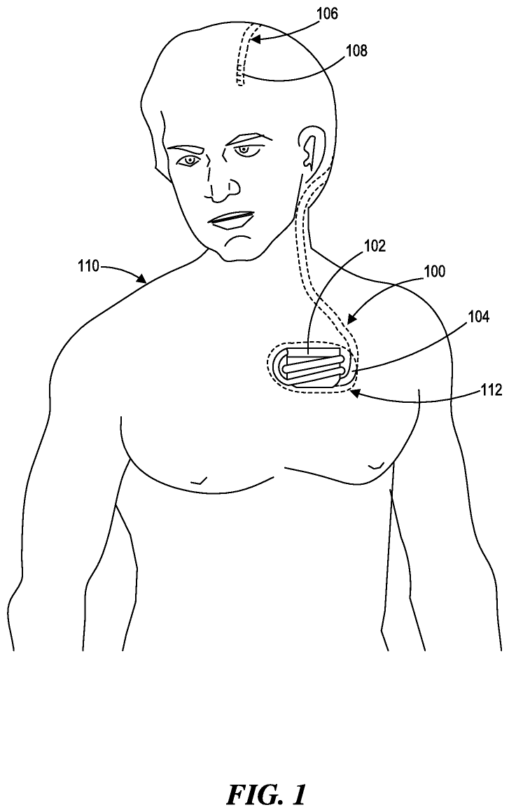

FIG. 1 shows a typical environment for embodiments of the anchoring system that utilizes deployable structures. In this example, an implantable medical system 100 is present within a body 110 of a patient. The implantable medical system 100 of this example includes an implanted device 102 that has been implanted into a subcutaneous or subfascial pocket 112. An implantable medical lead 104, or lead and extension combination, is routed between a target stimulation site and the implanted device 102.

In this example, the implanted device 102 is a neurostimulator configured for deep brain stimulation, as the lead 104 has a distal end 106 and distal electrodes 108 positioned within the brain. The location of convenience of the subcutaneous or subfascial pocket 112 in this example is in the upper pectoral region. However, it will be appreciated that embodiments of an anchoring system for an implanted device 102 are applicable to other types of devices and other locations of the target area and the subcutaneous or subfascial pocket. Other examples of implanted devices include but are not limited to neurostimulators for spinal cord stimulation where the lead 104 is directed to the spinal cord, peripheral nerve stimulation where the lead 104 is directed to a peripheral nerve, pelvic nerve stimulation where the lead 104 is directed to a pelvic nerve, cardiac stimulation where the lead 104 is directed to cardiac tissue, and the like as well as implantable drug pumps where a drug is delivered rather than stimulation. Other examples of locations of the subcutaneous or subfascial pocket include the upper buttocks area as well as other peripheral areas.

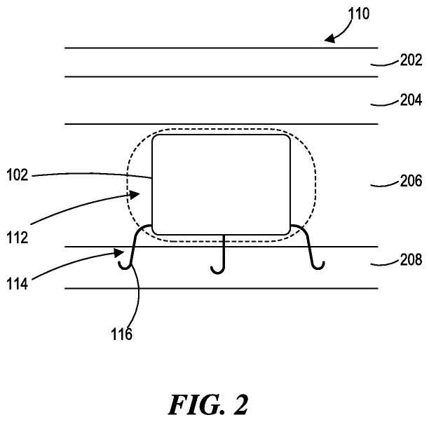

FIG. 2 shows an example of the subcutaneous pocket and surrounding layers of body tissue in more detail. The subcutaneous pocket may be formed in a subcutaneous fat layer 206 that resides beneath the epidermis layer 202 and dermis layer 204. The subcutaneous fat layer 206 resides above a layer of fascia 208. The implanted device 102 is positioned within the pocket 112 so that an anchoring system 114 with deployable structures 116 are adjacent the layer of fascia 208 so that when the deployable structures 116 are deployed, as shown, the deployable structures engage fascial tissue that forms the layer of fascia 208. This engagement of the deployable structures 116 of fascial tissue of layer 208 secure the position and orientation of the implanted device 102. While this subcutaneous pocket 112 is shown as one example of a tissue pocket 112, it will be appreciated that other tissue pocket locations are possible including subfascial.

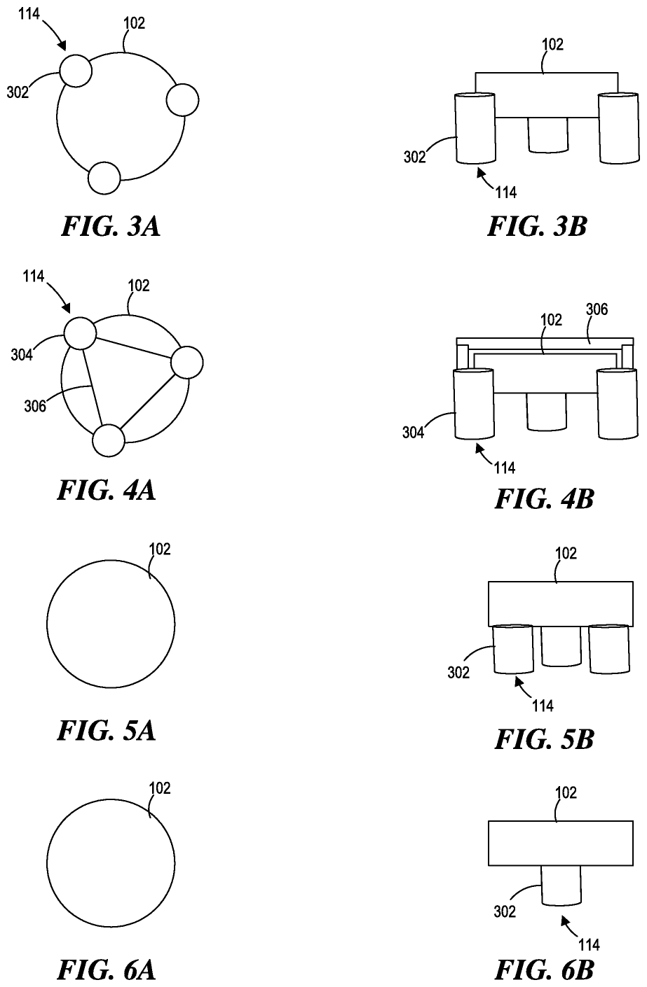

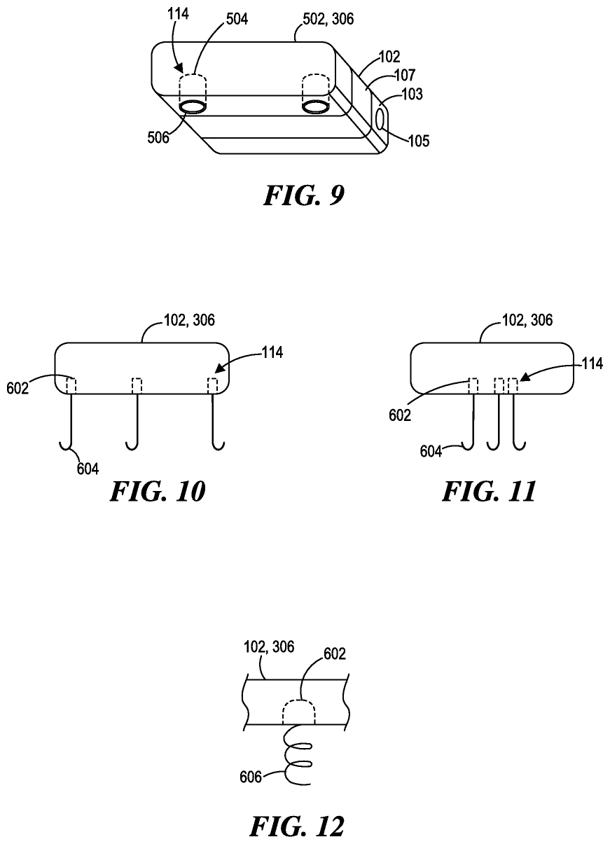

FIGS. 3A-6B show generalized views of the anchoring system 114 for purposes of illustrating examples of positioning and integration with the implanted device 102. In FIGS. 3A and 3B, it can be seen that multiple anchor points 302 including anchors and actuators of the anchors may be distributed about the periphery of the implanted device 102 by being an integral component of the implanted device. At each of the anchor points, 302 a deployable structure may be present so that once the implanted device 102 is within the pocket 112, the deployable structure at each anchor point 302 may then be deployed. The anchor points 302 may be formed in various ways, such as creating chambers distributed about the periphery of the outer body of the implanted device 102. Examples of deployable structures and actuators are discussed in more detail below with reference to FIGS. 9-27.

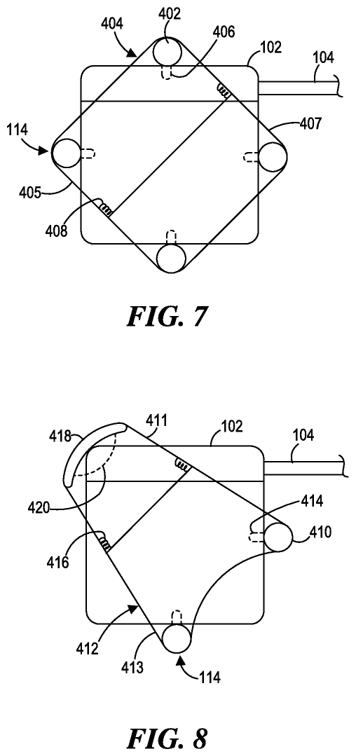

FIGS. 4A and 4B show a generalized example where the anchoring system 114 is not formed integrally with the implanted device 102 but instead has a separate anchoring system chassis 306 with one or more anchor points 304 including anchors and actuators of the anchors distributed about the periphery of the anchoring system chassis 306. This allows the anchoring system to be retrofitted to implanted devices that have been manufactured without integral anchoring systems. Furthermore, this allows a specific type of anchoring system to be chosen for a given implantation situation of an implanted device 102. The anchoring system chassis 306 may be added to the implanted device 102 in various ways including bonding via a glue or weld to an outer surface of the outer body of the implanted device 102. The anchoring system chassis 306 may be attached to the outer surface of the implanted device 102 in other ways such as frictional or interference fits, such as a snap fit or compression fit and the like. Examples are discussed in more detail below in relation to FIGS. 7 and 8.

FIGS. 5A and 5B show a generalized example where the anchoring system 114 is integral to the implanted device 104 but rather than being distributed about the periphery, may be positioned in a more concentrated cluster so as to provide a smaller footprint within the subcutaneous or subfascial pocket. As can be seen from the front view of FIG. 5A, in such a case the anchor points 302 are not visible as they reside entirely behind the implanted device 102.

FIGS. 6A and 6B show another generalized example where an anchoring system 114 may include a single anchoring point 302 that is positioned centrally on the implanted device 102. Such a configuration further minimizes the footprint of the anchoring system 114 within the subcutaneous or subfascial pocket.

FIG. 7 shows an example of an anchoring system 114 that is a separate structure from the implanted device 102. In this particular example, the anchoring system 114 includes a chassis 404 and at least four anchoring points 402 spaced about the chassis 404 with an anchor point 402 present at each of the four sides of the implanted device 102. The chassis 404 of this example includes two sections, a first section 405 and a second section 407 that are connected via a ratchet 408 that allows the sections 405 and 407 to be moved away from each other and to be moved back together. The anchoring system 114 also includes lip features 406 that may extend from the anchor points 402 or from other locations on the chassis 404. These lip features 406 engage the opposite side of the outer body of the implanted device 102 from the side that the sections 405 and 407 engage. The chassis 404 and lip features 406 may be constructed of materials such as titanium, ceramics, silicone, and other biostable materials.

The combination of the sections 405 and 407 and the lip features 406 allow the anchoring system 114 to be attached to the implanted device 102 as follows. The sections 405 and 407 may be ratcheted apart far enough to allow the implanted device 102 to pass between the lip features 406 and become seated against the sections 405 and 407. The sections 405 and 407 are ratcheted back together which then positions the lip features 406 into engagement with the opposite side of the implanted device 102 to thereby lock the anchoring system 114 to the implanted device 102 as shown in FIG. 7.

FIG. 8 shows another example of an anchoring system 114 that is a separate structure from the implanted device 102. In this particular example, the anchoring system 114 includes a chassis 412 and at least two anchoring points 410 on adjacent corners of the chassis 412. An attachment point 418, which may be another anchor point 402, a different type of anchor point than the anchor points 402, or may be just a coupling point, exists at an opposing corner from the anchor points 402. The chassis 412 of this example includes two sections, a first section 411 and a second section 413 that are connected via a ratchet 416 that allows the sections 411 and 413 to be moved away from each other and to be moved back together. The anchoring system 114 also includes lip features 414 on the anchor points 402 or elsewhere and a lip feature 420 at the attachment point 418. These lip features 414 and 418 engage the opposite side of the outer body of the implanted device 102 from the side that the sections 411 and 413 engage. The chassis 412 and lip features 414 and 418 may be constructed of materials including those discussed above for the example of FIG. 7.

The combination of the sections 411 and 413 and the lip features 414 and 418 allow the anchoring system 114 to be attached to the implanted device 102 as follows. The sections 411 and 413 may be ratcheted apart far enough to allow the implanted device 102 to pass between the lip features 414 and 418 and become seated against the sections 411 and 413. The sections 411 and 413 are ratcheted back together which then positions the lip features 414 and 418 into engagement with the opposite side of the implanted device 102 to thereby lock the anchoring system 114 to the implanted device 102 as shown in FIG. 8.

FIG. 9 shows an embodiment of the medical device 102 having a header 103 that defines an opening 105 and associated passageway for receiving an implantable medical lead 104. In this example, either a separate anchoring system chassis 306 is coupled to a portion 107 of the implanted device 102 or an integral anchoring system portion 502 is adjacent and permanently attached to the portion 107 of the implanted device 102. For instance, the portion 107 may be a hermetically sealed portion containing the necessary electronics to provide the stimulation signals to the lead 104 that is inserted into the opening 105.

The housing of the attached chassis 306 or the housing of the integral portion 502 may provide internal anchor point chambers 504 that are separate from the portion 107 and thereby are not required to be hermetically sealed. However, the chambers 504 may include wipers 506 that engage the deployable anchor that resides within the chamber 504 until being deployed. As the anchor is being deployed from the chamber 504 or being retracted into the chamber 504, the wipers 506 provide a degree of sealing engagement to the anchor, that may be of various forms such as a spine or barb discussed in more detail below. This sealing engagement reduces the amount of body fluid that ingresses into the chamber 504 where an actuator for the anchor may reside. The wipers 506 may be made from silicone or comparable material.

FIG. 10 shows an example of chambers 602, located either within an integral portion of the implanted device 102 or within a separate chassis 306, that contain deployable spines 604 about the periphery. FIG. 11 shows an example where the chambers 602 are more centrally located. In some embodiments, the spines 604 may also be retractable into the chambers 602. The spines 604 are anchors that when deployed engage the fascial tissue to fix the position of the implanted device 102 within the subcutaneous or fascial pocket. The spines 604 may be of various forms and shapes. For instance, the spines 604 may be hook shaped once deployed as shown in FIGS. 10 and 11. As an alternative, spines 606 may be spiral shaped once deployed as shown in FIG. 12.

One manner of providing the spines with a particular shape once deployed is to use a shape memory material. As one example, the spines may be constructed of a nickel-titanium shape memory alloy such as Nitinol. The spines 604, 606 may take on a different shape when retracted into the chamber, such as being rounded while wound on a spool, being linearized within a slot, or the like, but upon being deployed outside of the chamber 602, the spin regains the shape defined by the shape memory of the spine material.

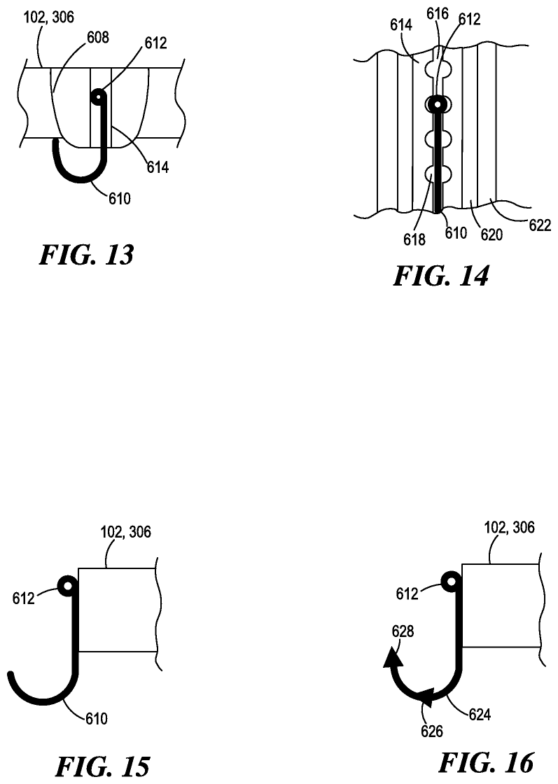

One example of a deployable and retractable spine implementation is shown in FIG. 13. Here the spine may reside within a linear guide 614 that formed within a chamber 608 for the spine 610 to reside within. The spine 610 may include a feature such as a thumb button 612 that allows a clinician to move the spine 610 up and down within the guide 614 of the chamber 608. A more detailed view of the linear guide 614 is shown in FIG. 14.

As shown in FIG. 14, the guide 614 defines a slot 616 that the spine 610 resides within. The slot includes features that interface with the feature of the spine, such as notches 618 that capture the button 612 formed on the end of the spine 610 to hold the spine in a fixed position. Manual force applied to the button 612 forces the button 612 to move out of a given notch and into an adjacent notch while the guide 614 continues to squeeze against the button 612, to thereby deploy or retract the spine 610 depending upon the direction of motion applied to the button 612. Outer rails 622 are present to provide a stable structure and are separated from the guide 614 by a gap 620 that allows the guide 614 to expand into the gap 620 when the button 612 is between notches 618. The guide 614 returns to the unexpanded shape when the button 612 rests within a given notch 618.

FIG. 15 shows an anchoring mechanism in the form of a hook shaped anchor 610, such as a spine or a hook, that resides on an outer side of either the implanted device 102 or separate chassis 306. This hook shaped anchor 610 also includes a button 612 that resides within a guide 614 as shown in FIG. 14 where the guide 614 resides on the outer surface of either the implanted device 102 or separate chassis 306. Thus, the hook shaped anchor 610 may also be deployed and retracted via manual control of the button 612. FIG. 16 shows another example of the hook shaped anchor 624 that includes barbs 626, 628 to provide for additional interference and friction when engaging the fascial tissue. As certain embodiments of these hook shaped anchors are positioned in an exterior position when retracted, rather than in a chamber, they shape may be maintained when retracted such that a shape memory material is not needed. Examples of materials used for such hook shaped anchors include stainless steel, titanium, and biostable rigid polymers such as nylon.

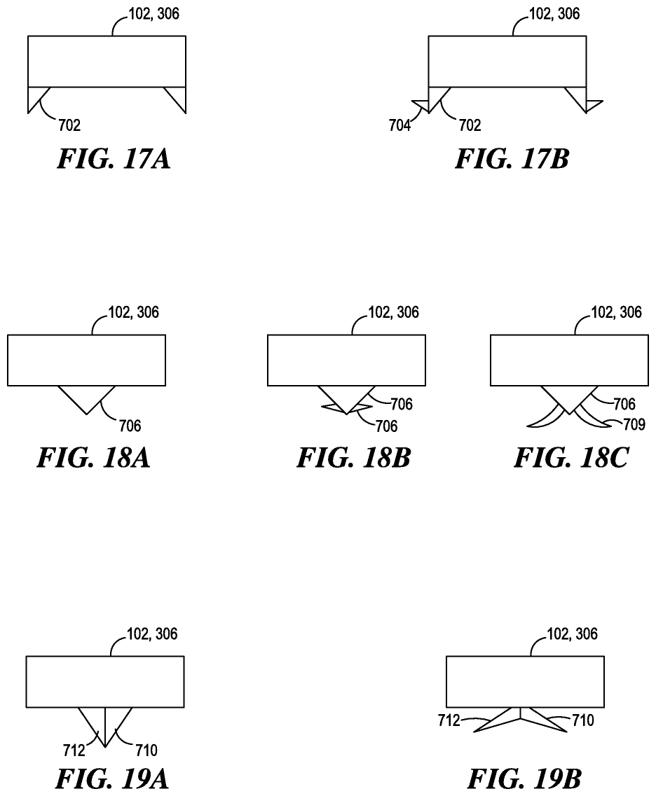

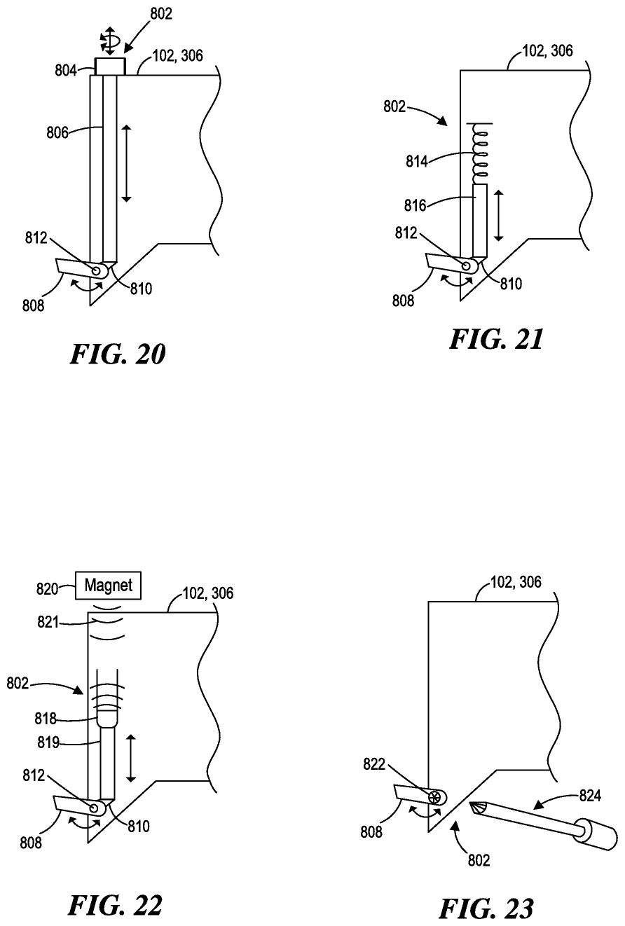

FIG. 17A shows an example of an implanted device 102 or separate chassis 306 that includes rigid spikes 702 that can pierce into the fascial tissue. However, to anchor the implanted device 102, these spikes 702 may include retractable barbs 704 as shown in FIG. 17B. Mechanisms for deploying and retracting these barbs 704 are discussed in more detail below starting at FIG. 20.

FIG. 18A shows an example of an implanted device 102 or separate chassis 306 that includes a central rigid spike 706 that can pierce into the fascial tissue. However, to anchor the implanted device 102, this spike 706 may include retractable barbs 708 as shown in FIG. 18B. Mechanisms for deploying and retracting these barbs 708 are discussed in more detail below starting at FIG. 20. FIG. 18C shows an alternative barb 709 that may be deployed from the spike 706. The barb 709 has saber shaped barb rather than a linear barb as in FIG. 18B.

FIG. 19A shows an example of an implanted device 102 or separate chassis 306 that includes rigid spikes 710 and 712 that are centrally located and can pierce into the fascial tissue. However, to anchor the implanted device 102, these spikes 710, 712 may include retractable barbs 711, 712 as shown in FIG. 19B. Mechanisms for deploying and retracting these barbs 711, 712 are also discussed in more detail below starting at FIG. 20.

FIG. 20 shows a first example of an actuator 802 that may also be used to deploy and/or retract the anchor 808. In this first example, the actuator 802 is in the form of a plunger that includes a control knob 802 and a control rod 806. The control knob 802 can be manipulated by a clinician as a triggering event to deploy or retract the anchor 808. In one scenario, the control knob 802 is a push-pull button 804 that forces the control rod 806 to extend the rod 806 to an extended position when the button is pushed as well as to retract the rod 806 to a retracted position when the button is pulled. Friction on the rod 806 may serve to hold the rod 806 in a fixed position when in either the deployed or retracted state.

In a different example, the control knob 802 may function by a rotating movement such as a screw that in turn moves the rod 806. The movement of the rod 806 is either in a direction toward the anchor 808 or a direction away from the anchor 808 depending upon whether the control knob 802 is turned in a clockwise or counterclockwise direction.

In either case, the end of the control rod 806 engages an attachment point 810 on the anchor 808. Thus, motion in a particular direction of the control rod 806 forces the anchor to move into the deployed or retracted position. In this particular example and from the perspective shown in FIG. 20, motion of the control rod 806 toward the anchor 808 produces a clockwise rotation of the anchor 808 while motion of the control rod 806 away from the anchor 808 produces a counter-clockwise rotation of the anchor 808. Also in this example, the anchor is attached to the implanted device 102 or chassis 306 via a hinge point 812 that allows the anchor 808 to pivot about the hinge point 812 when moving in the clockwise or counter-clockwise directions.

It will be appreciated that in one example, movement of the control rod 806 away from the anchor 808 which produces counterclockwise rotation retracts the anchor 808 while movement of the control rod 806 toward the anchor 808 which produces clockwise rotation deploys the anchor 808. It will also be appreciated that in another example, movement of the control rod 806 away from the anchor 808 which produces counterclockwise rotation deploys the anchor 808 while movement of the control rod 806 toward the anchor 808 which produces clockwise rotation retracts the anchor 808.

FIG. 21 shows a second example of an actuator 802 that may deploy and/or retract the anchor 808. In this example the actuator includes a spring 814 and control rod 816. The control rod 816 operates like the cam system of an internal spring-loaded tube of a ball point pen that extends and retracts by clicking the button on the end of the pen while holding the control rod 816 in a fixed position when not being clicked. A click button may be attached to the control rod 816 so that a triggering event is a clicking of the button by a clinician to cause the control rod 816 to transition between deployment and retraction states.

The end of the control rod 816 engages an attachment point 810 on the anchor 808. Thus, motion in a particular direction of the control rod 816 forces the anchor 808 to move into the deployed or retracted position. In this particular example and from the perspective shown in FIG. 21, motion of the control rod 816 toward the anchor 808 produces a clockwise rotation of the anchor 808 while motion of the control rod 816 away from the anchor 808 produces a counter-clockwise rotation of the anchor 808. The anchor 808 is attached to the implanted device 102 or chassis 306 via the hinge point 812 that allows the anchor 808 to pivot about the hinge point 812 when moving in the clockwise or counter-clockwise directions in the same manner as in the example of FIG. 20.

It will be appreciated that in one example of the configuration of FIG. 21, movement of the control rod 816 away from the anchor 808 which produces counterclockwise rotation retracts the anchor 808 while movement of the control rod 816 toward the anchor 808 which produces clockwise rotation deploys the anchor 808. It will also be appreciated that in another example, movement of the control rod 816 away from the anchor 808 which produces counterclockwise rotation deploys the anchor 808 while movement of the control rod 816 toward the anchor 808 which produces clockwise rotation retracts the anchor 808.

FIG. 22 shows a third example of an actuator 802 that may deploy and/or retract the anchor 808. In this example the actuator includes a magnetically sensitive component 818 and control rod 819. The control rod 819 is moved by the magnetically sensitive component 818. In one example, the magnetically sensitive component 818 is a magnet that is either attracted to or repelled from an external magnet 820 depending upon the orientation of the magnetic field 821 emanating from the external magnet 820. A triggering event occurs when a clinician brings an external magnet 20 in proximity to the magnetically sensitive component 818 to either produce an attractive force by the external magnet 20 that brings the control rod 819 away from the anchoring structure 808 or produce a repelling force by the external magnet 20 that forces the control rod 819 toward the anchoring structure 808. Friction on the rod 819 may serve to hold the rod 819 in a fixed position when in either the deployed or retracted state.

As another example, the magnetically sensitive component 818 may be a circuit that includes a magnetically sensitive element such as a Hall effect sensor or a reed switch. In either case, the presence of the external magnetic field 821 from the external magnet affects the Hall effect sensor or reed switch in such a way that the corresponding circuit of the component 818 activates an electric motor to move the control rod 819.

As in the prior examples, the end of the control rod 819 engages an attachment point 810 on the anchor 808. Thus, motion in a particular direction of the control rod 819 forces the anchor 808 to move into the deployed or retracted position. In this particular example and from the perspective shown in FIG. 22, motion of the control rod 819 toward the anchor 808 produces a clockwise rotation of the anchor 808 while motion of the control rod 819 away from the anchor 808 produces a counter-clockwise rotation of the anchor 808. The anchor 808 is attached to the implanted device 102 or chassis 306 via the hinge point 812 that allows the anchor 808 to pivot about the hinge point 812 when moving in the clockwise or counter-clockwise directions in the same manner as in the example of FIG. 20.

As with the other examples, it will be appreciated that in one example of the configuration of FIG. 22, movement of the control rod 819 away from the anchor 808 which produces counterclockwise rotation retracts the anchor 808 while movement of the control rod 819 toward the anchor 808 which produces clockwise rotation deploys the anchor 808. It will also be appreciated that in another example, movement of the control rod 819 away from the anchor 808 which produces counterclockwise rotation deploys the anchor 808 while movement of the control rod 819 toward the anchor 808 which produces clockwise rotation retracts the anchor 808.

FIG. 23 shows a fourth example of an actuator 802 that may deploy and/or retract the anchor 808. In this example, the anchor hinge point is established by a ratcheting screw 822 that is locked to the anchor 808. A triggering event occurs when the anchor 808 rotates about the hinge point due to a clinician using a mechanical tool 824 such as a screw driver with a tip that forms an engagement to the head of the ratcheting screw 822 to allow the clinician to apply a clockwise or counter-clockwise rotation to the ratcheting screw 822. This in turn causes clockwise or counterclockwise movement of the anchoring structure 808. Friction and/or interference within the ratcheting mechanism of the ratcheting screw 822 may serve to hold the ratcheting screw 822 in a fixed position when in either the deployed or retracted state.

It will be appreciated that in one example of the configuration of FIG. 23, counterclockwise rotation of the screw 822 retracts the anchor 808 while clockwise rotation deploys the anchor 808. It will also be appreciated that in another example, counterclockwise rotation of the screw 822 deploys the anchor 808 while clockwise rotation retracts the anchor 808.

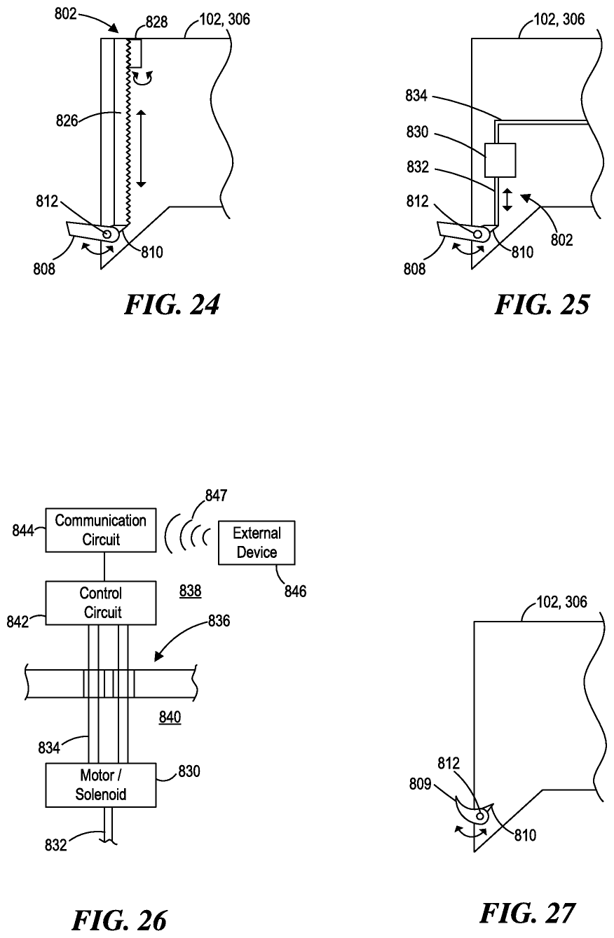

FIG. 24 shows a fifth example of an actuator 802 that may deploy and/or retract the anchor 808. The actuator 802 of this example includes a worm gear 828 where a triggering event occurs when a clinician manipulates the worm gear 828 to deploy or retract the anchor 808. For example, the worm gear 828 may have a portion that the clinician can grasp to turn the worm gear 828 or the worm gear 828 may have an interface where a mechanical tool used by the clinician creates an engagement to the worm gear 828. The worm gear 828 engages a rack 826 which is moved toward or away from the anchor 808, depending upon the direction the worm gear 828 is turned.

The end of the rack 826 engages an attachment point 810 on the anchor 808. Thus, motion in a particular direction of the rack 826 forces the anchor 808 to move into the deployed or retracted position. In this particular example and from the perspective shown in FIG. 24, motion of the rack 826 toward the anchor 808 produces a clockwise rotation of the anchor 808 while motion of the rack 826 away from the anchor 808 produces a counter-clockwise rotation of the anchor 808. Also in this example, the anchor is attached to the implanted device 102 or chassis 306 via the hinge point 812 that allows the anchor 808 to pivot about the hinge point 812 when moving in the clockwise or counter-clockwise directions. Friction on the rack 826, on the worm great 828, and/or within the worm gear 828 to rack 826 interface may serve to hold the rack 826 in a fixed position when in either the deployed or retracted state.

As with the prior examples, it will be appreciated that in one example, movement of the rack 826 away from the anchor 808 which produces counterclockwise rotation retracts the anchor 808 while movement of the rack 826 toward the anchor 808 which produces clockwise rotation deploys the anchor 808. It will also be appreciated that in another example, movement of the rack 826 away from the anchor 808 which produces counterclockwise rotation deploys the anchor 808 while movement of the rack 826 toward the anchor 808 which produces clockwise rotation retracts the anchor 808.

FIG. 25 shows a sixth example of an actuator 802 that may deploy and/or retract the anchor 808. The actuator 802 of this example includes a drive system 830 that can be electrically activated to deploy or retract the anchor 808. The drive system 830 includes an electric motor or solenoid coupled to a control rod 832, such as by a rack and pinion configuration, where the control rod 832 is moved toward or away from the anchor 808, depending upon the direction the electric motor of the drive system 830 turns, which is based on a control signal provided via electrical conductors 834. The ability to control the drive system 830 is discussed in more detail below with reference to FIG. 26.

The end of the control rod 832 engages an attachment point 810 on the anchor 808. Thus, motion in a particular direction of the control rod 832 forces the anchor 808 to move into the deployed or retracted position. In this particular example and from the perspective shown in FIG. 25, motion of the control rod 832 toward the anchor 808 produces a clockwise rotation of the anchor 808 while motion of the control rod 832 away from the anchor 808 produces a counter-clockwise rotation of the anchor 808. Also in this example, the anchor is attached to the implanted device 102 or chassis 306 via the hinge point 812 that allows the anchor 808 to pivot about the hinge point 812 when moving in the clockwise or counter-clockwise directions. Friction on the control rod 832 and/or within the drive system 830 may serve to hold the control rod 832 in a fixed position when in either the deployed or retracted state.

As with the prior examples, it will be appreciated that in one example, movement of the control rod 832 away from the anchor 808 which produces counterclockwise rotation retracts the anchor 808 while movement of the control rod 832 toward the anchor 808 which produces clockwise rotation deploys the anchor 808. It will also be appreciated that in another example, movement of the control rod 832 away from the anchor 808 which produces counterclockwise rotation deploys the anchor 808 while movement of the control rod 832 toward the anchor 808 which produces clockwise rotation retracts the anchor 808.

FIG. 26 shows an example of the control elements involve in deploying or retracting barbs using an electrical control signal. A triggering event may occur as a result of a wireless control signal 847 that is transmitted wirelessly by an external device 846, such as an external programmer used to program therapy parameters into the implanted device 102 or a separate external device. The clinician may enter an instruction into the external device 846, such as via a graphical user interface, that causes the wireless signal 847 to be sent that includes an external command to either deploy or retract the anchor 808. The wireless signal 847 may be a proximity based inductively coupled signal, an arm's length radio frequency signal, or a far field radio frequency signal such as a Medical Implant Communication Service (MICS) band or Bluetooth signal.

The implanted device 102 or chassis 306 may include circuitry within a hermetically sealed portion 838. The circuitry may include a communication circuit 844 that is capable of receiving at least one of a proximity based inductively coupled signal, arm's length radio frequency signal, or far field radio frequency signal that corresponds to the signal 847 being output from the external device 846. The communication circuit 844 provides the information from the signal 847 to a control circuit 842. The control circuit 842 may be of various forms such as hardwired analog and/or digital logic, a microcontroller, a general-purpose programmable processor, combinations thereof, and the like.

The electrical conductors 834 interconnect the control circuit 842 that is within the hermetically sealed portion 838 to a portion 840 that may be less than hermetically sealed, such as within an anchor deployment chamber like that shown in FIG. 9 or on an external location. The electrical conductors 834 carry the motor control signal generated by the control circuit 842 based on the signal 847 to the motor or solenoid 830. To allow the conductors 834 to pass from a hermetically sealed region 838 to the less than hermetically sealed region 840 where the motor or solenoid 830 is located without compromising the hermetic seal of the region 838, a feedthrough assembly 836 may be used. The feedthrough assembly 836 may be of the conventional nature, such as having metal ferrules welded in a hermetic fashion to the surrounding surface and filled with a non-conductive material such as glass that forms a hermetic seal to the conductors 834 and to the ferrule walls.

In the various actuators of FIGS. 20-25, the anchor 808 is shown as being a relatively liner barb. It will be appreciated that various other shapes may be used in conjunction with these and other actuators of the anchoring system. For instance, as shown in FIG. 29, the anchor may be a saber shaped barb 809 capable of being deployed and retracted in the manner discussed above.

Regardless of the shape of the anchor being deployed, it is desirable to protect the implantable medical lead or catheter 104 which may be somewhat vulnerable as the implantable medical lead 104 typically has a polymer jacket surrounding the internal conductors or catheter 104 typically has a polymer tubing forming a drug delivery lumen. Furthermore, it is often desirable to create strain relief for the implantable medical lead or catheter 104 by creating one or more strain relief loops of the implantable medical lead or catheter 104 within a subcutaneous or subfascial pocket. In scenarios where it is desirable to offer additional protection for the implantable medical lead or catheter 104 and to reduce the likelihood of damage due to deploying an anchor, a guide may be included in the subcutaneous or subfascial pocket so that the implantable medical lead or catheter 104 may be looped about the guide when creating the one or more strain relief loops to prevent the implantable medical lead or catheter 104 from inadvertently being in the path of the anchor being deployed.

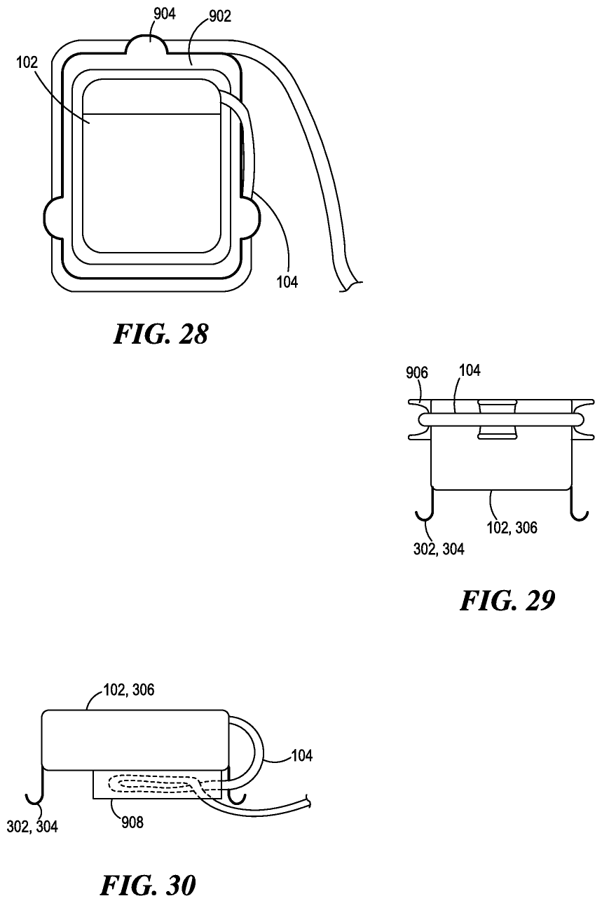

FIGS. 28-30 show examples of such guides. As shown in FIG. 28, a guide 902 is included around the periphery of the implanted device 102 within the subcutaneous or subfascial pocket. The guide 902 may reside in the pocket as a standalone object without being attached to the implanted device 102 or may alternatively have an attachment thereto via glue, bond, snap fit, and the like. The guide 902 allows the lead or catheter 104 to loop around while being held onto the guide 902 by the presence of retention lips 904 and the position of the lead or catheter 104 within the guide 902 separates the lead 104 from the area where the anchors are deployed.

FIG. 29 shows an example where the guide may take the form of one or more individual guides 906 mounted to the exterior of the housing of the implanted device or chassis 306. The one or more guides 906 may be machined into the exterior or attached such as by a weld, glue, or other bond. The lead or catheter 104 wraps about the implanted device 102 or chassis 306 while being retained within each of the individual guides 906. As with the guide 902, the position of the lead or catheter 104 within the guides 906 separates the lead 104 from the area where the anchors are deployed.

FIG. 30 shows an example where the guide is in the form of a shield 908 that forms a shield compartment where the one or more strain relief loops of the lead or catheter 104 may be contained. The shield 908 may be formed integrally with the exterior of the implanted device 102 or chassis 306 or may be attached via a weld, glue, or other bond. The shield 908 has at least one opening to allow the lead or catheter 104 to enter and exit the interior of the shield 908. As with the guides 902 and 906, the position of the lead or catheter 104 within the shield 908 as well as the position of the opening(s) for ingress and egress of the lead or catheter 104 separates the lead or catheter 104 from the area where the anchors are deployed.

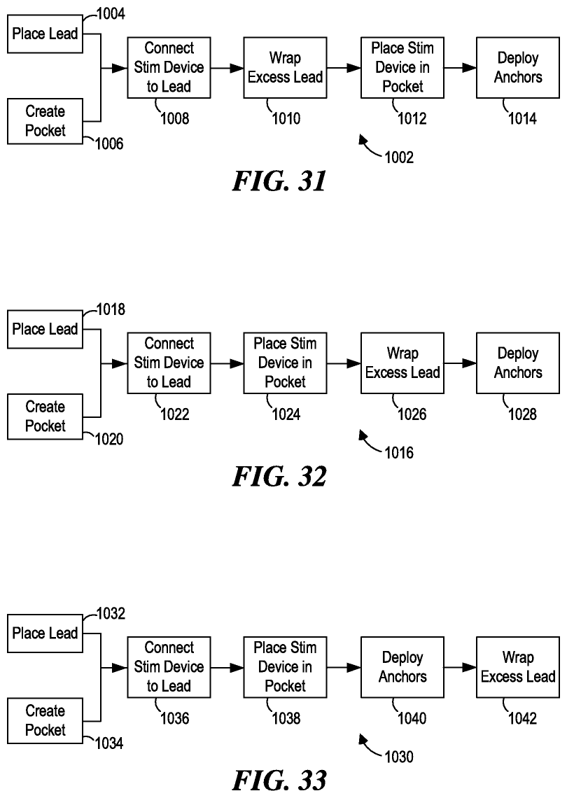

FIGS. 31-33 show examples of a set of steps that may be taken when implanting an implantable medical system 100 including an implanted device 102 and lead or catheter 104. In a first example 1002, the lead or catheter 104 is placed into the body and routed between the target stimulation site and the site where the implanted device 102 will be positioned at step 1004 contemporaneously with the subcutaneous or subfascial pocket being created where the implanted device 102 will be positioned at step 1006. The proximal end of the lead or catheter 104 may exit the body at the subcutaneous or subfascial pocket and this proximal end is connected to the implanted device 102 at step 1008 while the implanted device 102 remains outside of the body of the patient. The excess amount of lead or catheter may then be wrapped around the implanted device 102 to form the strain relief loop at step 1010. If a guide is present, such as one of the guides discussed above with reference to FIGS. 28-30, then the lead or catheter 104 is wrapped around the guide at the step 1010.

At this point, the implanted device 102 is ready to be implanted. The clinician places the implanted device 102 into the subcutaneous or subfascial pocket at step 1012. At this point, the clinician then deploys the one or more anchors of the anchoring system at step 1014 to anchor the implanted device 102 to the fascial tissue adjacent the subcutaneous or subfascial pocket. For example, the manner of deployment of the anchor may be in accordance with any of the examples of actuators discussed above. At some point after placement of the device 102 into the subcutaneous or subfascial pocket, the incision providing access to the subcutaneous or subfascial pocket can be closed. For instance, where the triggering of the deployment of the anchor involves manual manipulation by pressing a button, turning a knob, or using a tool to turn a screw, the pocket may be closed after the anchors are deployed. Where direct manual manipulation is not required, such as where a magnetic field is used or an external communication signal, then the pocket may be closed before or after deployment of the anchors.

FIG. 32 shows an alternate example 1016 of a set of steps that may be taken when implanting an implantable medical system 100 including the implanted device 102 and lead or catheter 104. The lead or catheter 104 is placed into the body and routed between the target stimulation site and the site where the implanted device 102 will be positioned at step 1018 contemporaneously with the subcutaneous or subfascial pocket being created where the implanted device 102 will be positioned at step 1020. The proximal end of the lead 104 may exit the body at the subcutaneous or subfascial pocket and this proximal end is connected to the implanted device 102 at step 1022 while the implanted device 102 remains outside of the body of the patient.

At this point, the implanted device 102 is ready to be implanted. The clinician places the implanted device 102 into the subcutaneous or subfascial pocket at step 1024. Then the excess amount of lead or catheter 104 may be wrapped around the implanted device 102 within the pocket to form the strain relief loop at step 1026. If a guide is present, such as one of the guides discussed above with reference to FIGS. 28-30, then the lead or catheter 104 is wrapped around the guide at the step 1026. At this point, the clinician then deploys the one or more anchors of the anchoring system at step 1028 to anchor the implanted device 102 to the fascial tissue adjacent the subcutaneous or subfascial pocket. For example, the manner of deployment of the anchor may be in accordance with any of the examples of actuators discussed above. At some point either before or after deployment of the anchors, depending upon whether they require manual manipulation of the anchoring system as the triggering event for deployment, the incision providing access to the subcutaneous or subfascial pocket can be closed.

FIG. 33 shows another alternate example 1030 of a set of steps that may be taken when implanting an implantable medical system 100 including the implanted device 102 and lead or catheter 104. The lead or catheter 104 is placed into the body and routed between the target stimulation site and the site where the implanted device 102 will be positioned at step 1032 contemporaneously with the subcutaneous or subfascial pocket being created where the implanted device 102 will be positioned at step 1034. The proximal end of the lead or catheter 104 may exit the body at the subcutaneous or subfascial pocket and this proximal end is connected to the implanted device 102 at step 1036 while the implanted device 102 remains outside of the body of the patient.

At this point, the implanted device 102 is ready to be implanted. The clinician places the implanted device 102 into the subcutaneous or subfascial pocket at step 1038. The clinician then deploys the one or more anchors of the anchoring system at step 1040 to anchor the implanted device 102 to the fascial tissue adjacent the subcutaneous or subfascial pocket. For example, the manner of deployment of the anchor may be in accordance with any of the examples of actuators discussed above. Then the excess amount of lead or catheter 104 may be wrapped around the implanted device 102 within the pocket to form the strain relief loop at step 1042. If a guide is present, such as one of the lead guides discussed above with reference to FIGS. 28-30, then the lead or catheter 104 is wrapped around the lead guide at the step 1026. At some point thereafter, the incision providing access to the subcutaneous or subfascial pocket can be closed.

For any of the examples of FIGS. 31-33, manual suturing of the implanted device 102 to the fascial tissue may be avoided while relying on the deployed anchors to provide the fixation within the pocket. However, if desired, manual suturing may also be done in addition to the deployed anchors at any point after the implanted device 102 has been placed into the pocket and prior to the incision at the pocket being closed.

Additionally, should there be reason to move the implanted device 102 within the pocket after deployment of the anchors, such as to reposition the implanted device 102 during the implantation procedure or at some point thereafter or should there be a need to explant the implanted device, the clinician may then retract the deployed anchors in any of the manners discussed above to allow the implanted device 102 to be moved within the pocket and/or removed from the pocket altogether.

While embodiments have been particularly shown and described, it will be understood by those skilled in the art that various other changes in the form and details may be made therein without departing from the spirit and scope of the invention.

What is claimed is:

1. An anchoring system for an implanted device of an implantable medical system, comprising:
an anchor configured to become affixed to fascial tissue adjacent a tissue pocket; and
an actuator coupled to the anchor, the actuator being responsive to a triggering event to move the anchor between a retracted position and a deployed position where the anchor becomes affixed to the fascial tissue once in the deployed position.

2. The anchoring system of claim 1, wherein the actuator is directly coupled to the implanted device.

3. The anchoring system of claim 2, wherein the implantable medical system comprises an implantable medical lead or catheter coupled to the implanted device.

4. The anchoring system of claim 2, further comprising an anchoring system chassis that is attached to a body of the implanted device, the actuator and the anchor being coupled to the anchoring system chassis.

5. The anchoring system of claim 4, wherein the anchoring system chassis is attached by a glue to the body of the implanted device.

6. The anchoring system of claim 4, wherein the anchoring system chassis is attached by a weld to the body of the implanted device.

7. The anchoring system of claim 4, wherein the anchoring system chassis is attached by a snap fit to the body of the implanted device.

8. The anchoring system of claim 4, wherein the anchoring system chassis ratchets into a compression fit to the body of the implanted device.

9. The anchoring system of claim 4, wherein the anchoring system chassis comprises a guide.

10. The anchoring system of claim 4, wherein the anchoring system chassis comprises a shield compartment.

11. The anchoring system 2, wherein the actuator and anchor are integral to the implanted device.

12. The anchoring system of claim 11, wherein the anchor resides within a chamber defined by a housing of the implanted device.

13. The anchoring system of claim 12, wherein the chamber includes a wiper that contacts the anchor.

14. The anchoring system of claim 11, wherein the implanted device comprises a guide that is integral to a housing of the implanted device.

15. The anchoring system of claim 11, wherein the implanted device comprises a shield compartment.

16. The anchoring system of claim 1, wherein the actuator is responsive to a first triggering event to move the anchor from a retracted position to a deployed position where the anchor becomes affixed to the fascial tissue adjacent the tissue pocket once in the deployed position and the actuator being responsive to a second triggering event that is subsequent to the first triggering event to move the anchor from the deployed position to the retracted position.

17. The anchoring system of claim 1, wherein the actuator comprises a spring-loaded actuator.

18. The anchoring system of claim 1, wherein the actuator comprises a magnetically sensitive actuator and wherein the triggering event comprises the presence of an external magnetic field that triggers the actuator.

19. The anchoring system of claim 18, wherein the actuator comprises a Hall effect sensor.

20. The anchoring system of claim 18, wherein the actuator comprises a reed switch.

21. The anchoring system of claim 1, wherein the actuator comprises a mechanical tool interface and wherein the triggering event comprises movement of the mechanical tool interface by a mechanical tool that is in engagement with the mechanical tool interface.

22. The anchoring system of claim 21, wherein the mechanical tool interface comprises a head of a screw and wherein the mechanical tool comprises a screwdriver.

23. The anchoring system of claim 22, wherein the actuator comprises a plunger with a first end coupled to a control knob and the anchor is attached at a hinge and coupled to a second end of the plunger so that operation of the control knob moves the plunger to thereby rotate the anchor about the hinge.

24. The anchoring system of claim 1, wherein the actuator is electrically controlled and comprises a control circuit and wherein the triggering event comprises an external command received at the control circuit that causes the control circuit to provide an electrical control signal.

25. The anchoring system of claim 24, wherein the actuator comprises an electric motor.

26. The anchoring system of claim 24, wherein the control circuit resides within a hermetically sealed portion of the implantable medical system and wherein the control signal exits the hermetically sealed portion through a feedthrough.

27. The anchoring system of claim 1, wherein the actuator is a feature on an end of the anchor that interfaces with a feature on the implanted device to hold the anchor in a fixed position and wherein the triggering event is manual force being applied to the feature on the anchor to move the anchor out of the fixed position.

28. The anchoring system of claim 1, wherein the actuator comprises a rack and worm gear.

29. The anchoring system of claim 1, wherein the anchor forms a hook shape.

30. The anchoring system of claim 29, wherein the anchor comprises a barb.

31. The anchoring system of claim 1, wherein the anchor is saber shaped.

32. The anchoring system of claim 1, wherein the anchor is spiral shaped.

33. An anchoring system for an implantable medical system that includes an implanted device, comprising:
an anchor configured to become affixed to tissue; and
an actuator coupled to the anchor, the actuator being responsive to a first triggering event to move the anchor from a retracted position to a deployed position where the anchor becomes affixed to the tissue once in the deployed position and the actuator being responsive to a second triggering event that is subsequent to the first triggering event to move the anchor from the deployed position to the retracted position, the actuator being coupled in a fixed position relative to the implanted device.

34. An anchoring system for an implantable medical system, comprising:
an anchor configured to become affixed to fascial tissue adjacent a tissue pocket; and
an actuator coupled to the anchor, the actuator being responsive to a first triggering event to move the anchor from a retracted position to a deployed position where the anchor becomes affixed to the fascial tissue once in the deployed position and the actuator being responsive to a second triggering event that is subsequent to the first triggering event to move the anchor from the deployed position to the retracted position.

35. A method of anchoring an implanted device that includes an anchoring system, comprising:
placing the implanted device within a tissue pocket;
attaching an implantable medical lead or catheter to the implanted device; and
after the placing the implanted device within the tissue pocket, deploying at least one anchor from the anchoring system into fascial tissue.

36. The method of claim 35, further comprising wrapping the implantable medical lead or catheter about a guide prior to placing the implanted device within the tissue pocket.

37. The method of claim 35, further comprising wrapping the implantable medical lead or catheter about a guide after placing the implanted device within the tissue pocket and prior to deploying the at least one anchor.

38. The method of claim 35, further comprising wrapping the implantable medical lead or catheter about a guide after deploying the at least one anchor.
